(19) United States
(12) Patent Application Publication
(10) Pub. No.: US 2004/0247630 A1
(43) Pub. Date: Dec. 9, 2004

(54) Cosmetic and/or pharmaceutical preparations

(76) Inventors: Werner Seipel, Hilden (DE); Mirella Nalborczyk, Titz (DE); Jessica Schneider, Koeln (DE)

Correspondence Address: COGNIS CORPORATION, PATENT DEPARTMENT, 300 BROOKSIDE AVENUE, AMBLER, PA, 19002, US

(21) Appl. No.: 10/492,309

(22) PCT Filed: Oct. 4, 2003
(86) PCT No.: PCT/EP02/11119

(30) Foreign Application Priority Data
Oct. 13, 2001 (DE) 10150729.1

(51) Int. Cl. A61K 7/06; A61K 7/11

(52) U.S. Cl. 424/401; 424/70.13

(57) ABSTRACT

A lipid-layer enhancing composition containing: (a) an alkyl and/or alkenyl oligoglycoside;(b) a fatty acid partial glyceride; (c) an alkylene glycol fatty acid ester; (d) optionally, a polymeric thickener; (e) optionally, a polyol; (f) optionally, water; (g) optionally, a cationic polymer, and wherein the composition is capable of providing an opacifying effect.

### FIELD OF THE INVENTION

This invention relates to opacifier concentrates containing selected sugar surfactants, fatty acid partial glycerides and alkylene glycol fatty acid esters and to their use as lipid layer enhancers for the production of cosmetic or pharmaceutical products.

### PRIOR ART

Cosmetic products are often produced with a flat white opaqueness using so-called opacifiers. Opacifiers are fine-particle polymer or solids dispersions which, besides water and/or a polyol, for example glycerol, essentially contain only a wax component and a suitable emulsifier. If such products are used in preparations for the cleaning and care of human skin and hair, their percentage content of surfactants is increased. The increased surfactant content would excessively dry out skin and hair, so that the use of lipid layer enhancers is recommended. It is obvious that not only must such substances have an adequate lipid-layer-enhancing effect, they are also expected to show optimal dermatological compatibility. At the same time, it is important for the dispersions to consist of very fine particles so that gradual sedimentation is prevented; in addition, a white opaqueness and no pearlescence should be developed.

Surfactant formulations often contain glyceryl monoalkyl esters in combination with alkyl polyglycosides as lipid layer enhancers, cf. for example German patents DE 41 39 935 C2 and DE 19543633 C2. In these formulations, however, the lipid-layer-enhancing effect is dominant. Their cleaning effect and their opacifying effect are both unsatisfactory.

German patent DE 195 11 572 C2 describes opacifier concentrates based on wax components with a solids content of 40 to 60% by weight which contain alk(en)yl oligoglycosides and partial glycerides in a ratio of 15:1 to 8:1. These known compositions have an inadequate lipid-layer-enhancing effect in rinse-off applications. The opacifier preparations based on wax components disclosed in DE 10034619 A1, which contain an emulsifier mixture of at least one alkyl and/or alkenyl oligoglycoside (a), at least one fatty acid partial glyceride (b) and optionally at least one amphoteric surfactant (c) in a ratio by weight of (a) and optionally (c) to (b) of 6:1 to 3:1, are distinguished by high stability and good processing properties. However, they also have an unfavorable ratio of cleaning effect and foam behavior to lipid-layer-enhancing properties.

Accordingly, the complex problem addressed by the present invention was to provide surfactant formulations with an opacifying effect which would combine a favorable cleaning effect with an optimal lipid-layer-enhancing effect and which would leave the skin with a pleasant feeling, would show high dermatological compatibility and would be easy to produce.

### DESCRIPTION OF THE INVENTION

The present invention relates to lipid-layer-enhancing and opacifying surfactant preparations containing

(a) 0.1 to 20% by weight alkyl and/or alkenyl oligoglycosides and

(b) 0.01 to 3% by weight fatty acid partial glycerides selected from the group consisting of oleic acid monoglyceride, oleic acid diglyceride, isostearic acid monoglyceride, isostearic acid diglyceride, behenic acid monoglyceride, behenic acid diglyceride, isobehenic acid monglyceride and/or isobehenic acid diglyceride,

(c) 0.05 to 10% by weight alkylene glycol fatty acid esters and optionally

(d) 0 to 3% by weight polymeric thickeners and optionally

(e) 0 to 5% by weight polyols and optionally

(f) 0 to 1% by weight cationic polymers,

with the proviso that the quantities shown add up to 100% by weight, optionally with other auxiliaries and additives and/or water.

It has surprisingly been found that surfactant formulations with the composition shown above have a storage-stable opacifying effect and, despite favorable cleaning properties, high foam stability and rapid foaming kinetics, show an excellent lipid-layer-enhancing effect. After application, they leave the skin with a pleasant feeling and are distinguished by high dermatological compatibility. When used on the hair, they lead to a distinct improvement in wet combability, to a pleasant softness and to distinctly improved luster. In addition, the formulations can readily be produced without heat.

Surfactants

Suitable surfactants are anionic, nonionic and/or amphoteric or zwitterionic surfactants which are normally present in the preparations in quantities of about 0.1 to 70, preferably 0.5 to 50 and more particularly 0.5 to 40% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride(ether)sulfates, fatty acid amide(ether)sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl(ether)phosphates.

If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, fatty acid glucamides, alkylamidobetaines, amphoacetals and/or protein fatty acid condensates, preferably based on wheat proteins.

Alkyl and/or Alkenyl Oligoalycosides

Alkyl and alkenyl oligoglycosides which are used as the sugar surfactant component (a1) are known nonionic surfactants which correspond to formula (I):

R¹O-[G]ₚ (I)

in which R¹ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry, for example by acid-catalyzed acetalization of glucose with fatty alcohols.

The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (I) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view.

The alkyl or alkenyl group R¹ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl radical R⁹ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} cocoalcohol with a DP of 1 to 3 are preferred.

Fatty Acid Partial Glycerides

The selected fatty acid partial glycerides which form component (b) are known substances which may be prepared by the relevant methods of preparative organic chemistry. Fatty acid partial glycerides, i.e. generally technical mixtures of mono- and diglycerides, are normally obtained by transesterification of the corresponding triglycerides with glycerol or by the selective esterification of fatty acids. The removal of unreacted starting materials and the concentration of monoglycerides in the mixtures are generally achieved by molecular distillation. The partial glycerides of the present invention are preferably prepared by esterification of glycerol with oleic acid, isostearic acid, behenic acid or isobehenic acid. The isobehenic acid is the hydrogenated monomer fraction which is obtained in the dimerization of erucic acid. The invention includes the observation that technical mono/diglyceride mixtures show better dermatological compatibility in use than the pure monoglycerides. Accordingly, technical fatty acid mono-/diglycerides with a molar ratio of mono- to diester of 10:90 to 90:10 and more particularly 80:20 to 50:50 are preferred. Fatty acid partial glycerides of mixtures of saturated and unsaturated fatty acids have proved to be particularly effective in the preparations and are preferably present in a ratio of 1:1.

Alkylene Glycol Fatty Acid Esters

The alkylene glycol fatty acid esters used are compounds corresponding to formula (II):

R²CO—O-[A]-O—R³ (I)

in which R²CO is an aliphatic acyl group containing 6 to 22 carbon atoms and 0 and/or 1, 2 or 3 double bonds, R³ has the same meaning as R²CO or is a hydroxyl group and A is a linear or branched, optionally hydroxysubstituted alkylene group containing 2 to 5 carbon atoms.

These waxes are preferably esters of ethylene glycol or propylene glycol with caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. The use of ethylene glycol distearate is particularly preferred.

Polymeric Thickeners

Suitable polymeric thickeners are, for example, polysaccharides, more especially xanthan gum, guar-guar, agar-agar, alginates and cellulose esters, such as methyl cellulose, carboxymethyl cellulose, hydroxyethyl and hydroxypropyl cellulose, also polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone.

Polyols

Polyols suitable for the purposes of the invention preferably contain 2 to 15 carbon atoms and at least two hydroxyl groups. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

sugar alcohols containing 5 to 12 carbon atoms, for example sorbitol or mannitol;

sugars containing 5 to 12 carbon atoms, for example glucose or sucrose;

amino sugars, for example glucamine.

Cationic Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Commercial Applications

The preparations according to the invention are distinguished by high dermatological compatibility, good cleaning properties and an excellent lipid-layer-enhancing effect.

Embodiments of the lipid-layer-enhancing and opacifying surfactant preparations according to the invention contain

(a) 0.1 to 20% by weight alkyl and/or alkenyl oligoglycosides and

(b) 0.01 to 3% by weight fatty acid partial glycerides selected from the group consisting of oleic acid monoglyceride, oleic acid diglyceride, isostearic acid monoglyceride, isostearic acid diglyceride, behenic acid monglyceride, behenic acid diglyceride, isobehenic acid monglyceride and/or isobehenic acid diglyceride,

(c) 0.05 to 10% by weight alkylene glycol fatty acid esters and optionally

(d) 0 to 3% by weight polymeric thickeners and optionally

(e) 0 to 5% by weight polyols and optionally

(f) 0 to 1% by weight cationic polymers,

more particularly

(a) 0.1 to 10% by weight alkyl and/or alkenyl oligoglycosides and

(b) 0.05 to 1% by weight fatty acid partial glycerides selected from the group consisting of oleic acid monoglyceride, oleic acid diglyceride, isostearic acid monoglyceride, isostearic acid diglyceride, behenic acid monglyceride, behenic acid diglyceride, isobehenic acid monglyceride and/or isobehenic acid diglyceride,

(c) 0.1 to 5% by weight alkylene glycol fatty acid esters and optionally

(d) 0 to 2% by weight polymeric thickeners and optionally

(e) 0 to 3% by weight polyols and optionally

(f) 0 to 0.5% by weight cationic polymers,

preferably

(a) 0.5 to 5% by weight alkyl and/or alkenyl oligoglycosides and

(b) 0.1 to 0.5% by weight fatty acid partial glycerides selected from the group consisting of oleic acid monoglyceride, oleic acid diglyceride, isostearic acid monoglyceride, isostearic acid diglyceride, behenic acid monglyceride, behenic acid diglyceride, isobehenic acid monglyceride and/or isobehenic acid diglyceride,

(c) 0.5 to 3% by weight alkylene glycol fatty acid esters and optionally

(d) 0 to 3% by weight polymeric thickeners and optionally

(e) 0 to 5% by weight polyols and optionally

(f) 0 to 1% by weight cationic polymers,

more preferably

(a) 0.5 to 5% by weight alkyl and/or alkenyl oligoglycosides and

(b) 0.1 to 0.5% by weight fatty acid partial glycerides selected from the group consisting of oleic acid monoglyceride, oleic acid diglyceride, isostearic acid monoglyceride, isostearic acid diglyceride, behenic acid monglyceride, behenic acid diglyceride, isobehenic acid monglyceride and/or isobehenic acid diglyceride,

(c) 0.5 to 3% by weight alkylene glycol fatty acid esters and optionally

(d) 0.1 to 1% by weight polymeric thickeners and optionally

(e) 0 to 5% by weight polyols and optionally

(f) 0.05 to 0.5% by weight cationic polymers,

with the proviso that the quantities shown add up to 100% by weight, optionally with other auxiliaries and additives and/or water.

The present invention also relates to the use of preparations containing

(a) alkyl and/or alkenyl oligoglycosides and

(b) fatty acid partial glycerides selected from the group consisting of oleic acid monoglyceride, oleic acid diglyceride, isostearic acid monoglyceride, isostearic acid diglyceride, behenic acid monglyceride, behenic acid diglyceride, isobehenic acid monglyceride and/or isobehenic acid diglyceride and

(c) ethylene glycol distearate,

with the proviso that the ratio by weight of (a) to (b) is between 6:1 and 2:1 and the ratio by weight of (c) to (b) is between 12:1 and 7:1, as lipid layer enhancers in cosmetic and pharmaceutical products.

The care preparations mentioned, for example hair shampoos, shower baths, foam baths, may contain oil components, emulsifiers, silicone compounds, biogenic agents, antidandruff agents, film formers, preservatives, perfume oils, dyes and the like as further auxiliaries and additives.

Oil Components

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di-and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on fatty alcohols containing 6 to 18 and preferably 8 to 10 carbon atoms, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

addition products of 1 to 15 mol ethylene oxide onto castor oil and/or hydrogenated castor oil;

addition products of 15 to 60 mol ethylene oxide onto castor oil and/or hydrogenated castor oil;

partial esters of sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;

mixed esters of pentaerythritol, fatty acids, citric acid and fatty alcohol and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethylene glycol-30 Dipolyhydroxystearate;

polymer emulsifiers, for example Pemulen types (TR-1, TR-2) of Goodrich;

polyalkylene glycols and

glycerol carbonate.

Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as lipid layer enhancers for cosmetic formulations.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are aliphatic fatty acids containing 12 to 22 carbon atoms, such as for example palmitic acid, stearic acid or behenic acid, and dicarboxylic acids containing 12 to 22 carbon atoms, such as azelaic or sebacic acid for example.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

Biogenic Agents

Biogenic agents in the context of the invention are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract and bambara nut extract, and vitamin complexes.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetik-verordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat. Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture as a whole.

### EXAMPLES

### I. Application Examples

Lipid-layer-enhancing behavior was evaluated by testing the wet combability of hair tresses treated with lipid layer enhancers. To this end, the tresses were medium-bolded before the blank measurement. After a contact time of 5 mins., the test formulations (1 g/1 g hair) were rinsed for 1 min. under standard conditions (38° C., 1 liter/min.), The measurement was carried out on 20 tresses. Formulations 1 and 2 correspond to the invention; Example C1 is intended for comparison. The results are set out in Table 1.

Table I

Formulations for Wet Combability Measurements (Quantities as % by Weight)

Silicone-free hair shampoo based on wax dispersions (with polymeric thickener)

| Trade Name | INCI Name | C1 | 1 | 2 |
|---|---|---|---|---|
| Texapon ® N70 | Sodium Laureth Sulfate | 14.00 | 14.00 | 14.00 |
| Dehyton ® K | Cocoamidopropyl Betaine | 8.00 | 8.00 | 8.00 |
| Plantcare ® 818 UP | Coco Glycoside | 4.00 | 4.00 | 4.00 |
| Lipid layer enhancer* | | 0.00 | 5.00 | 10.00 |
| Euxyl ® K 400 | Methyldibromo Glutaronitrile | 0.10 | 0.10 | 0.10 |
| | and Phenoxyethanol | | | |
| Cosmedia Guar ® C 261 N | Guar Hydroxypropyl | 0.10 | 0.10 | 0.10 |
| | Trimonium Chloride | | | |
| Methocel ® E4M Premium | Hydroxpropyl | 1.10 | 1.10 | 1.10 |
| EP | Methylcellulose | | | |
| Citric acid/NaOH | | q.s. for pH 5.5 | | |
| Water | | to | to | to |
| | | 100.0 | 100.0 | 100.0 |
| Residual wet compatibility | | 83% | 73% | 50% |

*lipid-layer-enhancing component:
21.5% by weight glycol
7.5% by weight Coco Glyceride
1.5% by weight Glyceryl Oleate
1.5% by weight Glyceryl Stearate

Besides improved wet combability, formulations 1 and 2 produce a pleasant feel and increased luster of the dried hair in a half-head test.

To evaluate performance properties, four formulations according to the invention (1 to 5) were compared with three comparison formulations (C1 to C3) of conventional composition in regard to their physicochemical properties and their effect on the hair. The improved lipid-layer-enhancing effect on the hair of formulations 1 to 4 is particularly noticeable; this effect can be further improved by additional cationic polymers.

TABLE 2
Formulations for evaluating performance properties (quantities as % by weight)
Silicone-free hair shampoo based on wax dispersions

| Trade Name | INCI Name | C1 | C2 | C3 | 1 | 2 | 3 | 4 | 5 |
|---|---|---|---|---|---|---|---|---|---|
| Texapon ® NSO | Sodium Laureth Sulfate | 33.3 | 33.3 | 33.3 | 33.3 | 33.3 | 33.3 | 33.3 | 33.3 |
| Dehyton ® K | Cocamidopropyl Betaine | 9.8 | 9.8 | 9.8 | 9.8 | 9.8 | 9.8 | 9.8 | 9.8 |
| Plantacare ® 818 | Coco Glyceride | 2.5 | 2.5 | 2.5 | 1.25 | − | 1.25 | − | 2.5 |
| Lytron ® 631 (Morton | Sodium Styrene/Acrylates | 1.0 | 1.0 | 3.0 | − | − | − | − | − |
| International) | Copolymer (and) Sodium | | | | | | | | |
| | Lauryl Sulfate (and) | | | | | | | | |
| | Trideceth-7 | | | | | | | | |
| Lipid layer enhancer* | | − | − | − | 5.0 | 10.0 | 5.0 | 10.0 | 3.0 |
| Cosmedia Guar ® | Guar Hydroxypropyl | 0.2 | − | 0.2 | 0.2 | 0.2 | − | − | 0.2 |
| C261N | Trimonium Chloride | | | | | | | | |
| Perfume oil | | 0.3 | 0.3 | 0.3 | 0.3 | 0.3 | 0.3 | 0.3 | 0.3 |
| Euxyl ® K400 | Methyldibromo Gutaronitrile | 0.1 | 0.1 | 0.1 | 0.1 | 0.1 | 0.1 | 0.1 | 0.1 |
| | (and) Phenoxyethanol | | | | | | | | |
| Glycerin | Glycerin | 2.0 | 2.0 | 2.0 | 2.0 | 2.0 | 3.0 | 3.0 | 2.0 |
| NaCl | | 1.06 | 1.14 | 1.2 | 0.99 | 0.88 | 1.12 | 1.04 | 1.14 |
| Water | | to 100% | | | | | | | |
| Citric acid | | for pH adjustment | | | | | | | |
| pH value | | 5.0 | 5.1 | 5.5 | 5.3 | 5.2 | 5.1 | 5.0 | 5.4 |
| Viscosity mPas | | 6000 | 5600 | 5800 | 5000 | 4900 | 4500 | 5100 | 5200 |

−− no conditioning, poor combability
− no conditioning
+ conditioning is noticeable
++ conditioning is pronounced
+++ very good combability
*lipid-layer-enhancing component:
21.5% by weight glycol
7.5% by weight Coco Glyceride
1.5% by weight Glyceryl Oleate
1.5% by weight Glyceryl Stearate

1-10 (cancelled).

11. A lipid-layer enhancing composition comprising:
(a) an alkyl and/or alkenyl oligoglycoside;
(b) a fatty acid partial glyceride;
(c) an alkylene glycol fatty acid ester;
(d) optionally, a polymeric thickener;
(e) optionally, a polyol;
(f) optionally, water;
optionally, a cationic polymer, and

12. The composition of claim 11 wherein (a) is present in the composition in an amount of from about 0.1 to 20% by weight, based on the weight of the composition.

13. The composition of claim 11 wherein (a) is present in the composition in an amount of from about 0.5 to 5% by weight, based on the weight of the composition.

14. The composition of claim 11 wherein (b) is selected from the group consisting of an oleic acid monoglyceride, an oleic acid diglyceride, an isostearic acid monoglyceride, an isostearic acid diglyceride, a behenic acid monoglyceride, a behenic acid diglyceride, an isobehenic acid monoglyceride, an isobehenic acid diglyceride, and mixtures thereof.

15. The composition of claim 11 wherein (b) is derived from a mixture of saturated and unsaturated fatty acids in a ratio of 1:1.

16. The composition of claim 11 wherein (b) is present in the composition in an amount of from about 0.01 to 3% by weight, based on the weight of the composition.

17. The composition of claim 11 wherein (b) is present in the composition in an amount of from about 0.1 to 0.5% by weight, based on the weight of the composition.

18. The composition of claim 11 wherein (c) is present in the composition in an amount of from about 0.05 to 10% by weight, based on the weight of the composition.

19. The composition of claim 11 wherein (c) is present in the composition in an amount of from about 0.5 to 3% by weight, based on the weight of the composition.

20. A lipid-layer enhacing composition comprising:
(a) an alkyl and/or alkenyl oligoglycoside;
(b) a fatty acid partial glyceride; and
(c) an ethylene glycol distearate, and
wherein (a) and (b) are present in the composition in a ratio by weight of from about 6:1 to 2:1, and (b) and (c) are present in the composition in a ratio by weight of from about 1:12 to 1:7.

21. A process for treating human skin and/or hair comprising contacting the skin and/or hair with a lipid-enhancing composition containing:
(a) an alkyl and/or alkenyl oligoglycoside;
(b) a fatty acid partial glyceride;
(c) an alkylene glycol fatty acid ester;
(d) optionally, a polymeric thickener;
(e) optionally, a polyol;
(f) optionally, water;
(g) optionally, a cationic polymer, and
wherein the composition is capable of providing an opacifying effect.

22. The process of claim 21 wherein (a) is present in the composition in an amount of from about 0.1 to 20% by weight, based on the weight of the composition.

23. The process of claim 21 wherein (a) is present in the composition in an amount of from about 0.5 to 5% by weight, based on the weight of the composition.

24. The process of claim 21 wherein (b) is selected from the group consisting of an oleic acid monoglyceride, an oleic acid diglyceride, an isostearic acid monoglyceride, an isostearic acid diglyceride, a behenic acid monoglyceride, a behenic acid diglyceride, an isobehenic acid monoglyceride, an isobehenic acid diglyceride, and mixtures thereof.

25. The process of claim 21 wherein (b) is derived from a mixture of saturated and unsaturated fatty acids in a ratio of 1:1.

26. The process of claim 21 wherein (b) is present in the composition in an amount of from about 0.01 to 3% by weight, based on the weight of the composition.

27. The process of claim 21 wherein (b) is present in the composition in an amount of from about 0.1 to 0.5% by weight, based on the weight of the composition.

28. The process of claim 21 wherein (c) is present in the composition in an amount of from about 0.05 to 10% by weight, based on the weight of the composition.

29. The process of claim 21 wherein (c) is present in the composition in an amount of from about 0.5 to 3% by weight, based on the weight of the composition.

30. The process of claim 21 wherein the composition contains:
(a) an alkyl and/or alkenyl oligoglycoside;
(b) a fatty acid partial glyceride; and
(c) an ethylene glycol distearate, and
wherein (a) and (b) are present in the composition in a ratio by weight of from about 6:1 to 2:1, and (b) and (c) are present in the composition in a ratio by weight of from about 1:12 to 1:7.
